(19) United States
(12) Patent Application Publication
(10) Pub. No.: US 2018/0147175 A1
(43) Pub. Date: May 31, 2018

(54) COMPOSITIONS AND METHODS FOR TREATMENT OF MOVEMENT DISORDERS

(71) Applicant: ULTRAGENYX PHARMACEUTICAL INC., Novato, CA (US)

(72) Inventor: Fanny MOCHEL, Paris (FR)

(21) Appl. No.: 15/878,373

(22) Filed: Jan. 23, 2018

Related U.S. Application Data
(63) Continuation of application No. 15/092,883, filed on Apr. 7, 2016, now abandoned.
(60) Provisional application No. 62/234,860, filed on Sep. 30, 2015.
(60) Provisional application No. 62/144,036, filed on Apr. 7, 2015.

(51) Int. Cl. A61K 31/225; A61K 31/19

(52) U.S. Cl. CPC A61K 31/225; A61K 31/19

(57) ABSTRACT

The present invention relates to the treatment and prevention of movement disorders with the administration of one or more propionyl-CoA precursors.

### CROSS-REFERENCE TO RELATED APPLICATIONS

The present application claims priority to, and the benefit of U.S. Provisional Application Nos. 62/144,036, filed Apr. 7, 2015, and 62/234,860, filed Sep. 30, 2015, each of which is herein incorporated by reference in its entirety.

### FIELD OF THE INVENTION

The present invention relates to the treatment and prevention of movement disorders.

### BACKGROUND OF THE INVENTION

Glucose transporter type 1 deficiency syndrome (GLUT1-DS) is caused by impaired glucose transport across the blood-brain barrier and into astrocytes, leading to cerebral energy deficiency. GLUT1-DS is due to disrupted SLC2A1 activity, such as mutations in the SLC2A1 gene encoding the glucose transporter GLUT1. The phenotype typically comprises psychomotor retardation and permanent motor disorders, associated with paroxysmal manifestations including seizures and non-epileptic paroxysmal episodes (Pons et al., 2010, Mov Disord. 25: 275-281). With age, seizures tend to become less prominent whereas the frequency of non-epileptic paroxysmal episodes increases (Gras et al., 2014, Rev Neurol (Paris) 170(2): 91-99). In patients with milder forms of the disease, paroxysmal movement disorders, especially dyskinesia and dystonic episodes, may be the main or the sole manifestations of the disease and can occur at any age. Dyskinesia may be induced by exercise, i.e., paroxysmal exercise-induced dyskinesia (known as PED) (Schneider et al., 2009, Mov Disord. 24: 1684-8). Ketogenic diets, which provide ketone bodies to the brain and compensate for the lack of glucose, are efficient in controlling seizures in GLUT1-DS, but less effective in controlling movement disorders. Moreover, many patients—especially adolescents and adults—have difficulties complying with the difficult constraints of these long-term diets and their side effects.

The present invention stems from the finding that triheptanoin, an odd-chain triglyceride, dramatically improves paroxysmal movement disorders in GLUT1-DS patients with non-epileptic paroxysmal manifestations. Unlike even-chain fatty acids metabolized to acetyl-CoA only, the odd-chain triglyceride triheptanoin can provide both acetyl-CoA and propionyl-CoA, two key carbon sources for the Krebs cycle. Without being bound by theory, it is believed that the striking clinical response described herein is attributed to the production of propionyl-CoA that stems from the catabolism of triheptanoin and the concomitant production of C5-ketone bodies.

### SUMMARY OF THE INVENTION

The present invention relates to compositions that are useful in the treatment of movement disorders and methods of using said compositions to treat and/or prevent movement disorders.

The present invention is based, in part, on the discovery that the propionyl-CoA precursor, such as triheptanoin, has a significant therapeutic effect in GLUT1-DS patients suffering from movement disorders. The clinical response described herein was associated with the significant production of C5-ketone bodies and the normalization of f-MRS bioenergetics profile during brain activation.

Therefore, in one aspect, the invention relates to a method of treating a subject with a movement disease, disorder, or condition, wherein said method comprises the step of administering a therapeutically effective amount of at least one precursor of propionyl-CoA. In some embodiments, the administration provides a statistically significant therapeutic effect for the treatment of the movement disease, disorder, or condition.

In some embodiments, the administration of one or more propionyl-CoA precursors results in a reduction in paroxysmal manifestations associated with GLUT1-DS. In some embodiments, the number of paroxysmal manifestations following administration of one or more propionyl-CoA precursors is reduced by at least about 5%, 10%, 15%, 20%, 25%, 30%, 35%, 40%, 50%, 60%, 70%, 80%, 90%, or 95%. In some embodiments, administration of one or more propionyl-CoA precursors results in a statistically significant reduction in paroxysmal manifestations associated with GLUT1-DS.

In some embodiments, the administration of one or more propionyl-CoA precursors results in a reduction in dystonic events associated with GLUT1-DS. In some embodiments, the number of dystonic events following administration of one or more propionyl-CoA precursors is reduced by at least about 5%, 10%, 15%, 20%, 25%, 30%, 35%, 40%, 50%, 60%, 70%, 80%, 90%, or 95%. In some embodiments, administration of one or more propionyl-CoA precursors results in a statistically significant reduction in dystonic events associated with GLUT1-DS.

In some embodiments, the incidence of at least one clinical symptom associated with a GLUT1-DS mediated movement disorder is reduced by at least 10%, 20%, 40%, 60%, or 80% lower following administration of at least one propionyl-CoA precursor in a group of subjects.

In some embodiments, the precursor of propionyl-CoA is administered in the absence of a ketogenic diet.

In some embodiments, the precursor of propionyl-CoA is selected from an uneven chain fatty acid, a triglyceride, a C5 ketone body, a phospholipid, a branched amino acid and combinations thereof.

In certain exemplary embodiments, the precursor of propionyl-CoA is a triglyceride or phospholipid of uneven chain fatty acids.

In one exemplary embodiment, the precursor of propionyl-CoA is triheptanoin.

In some embodiments, the at least one precursor of propionyl-CoA is provided to the subject in an amount comprising at least about 20%, 25%, 30%, 35%, or at least about 40% of the dietary caloric intake for the subject.

In some embodiments, the movement disease, disorder or condition is associated with a glucose transporter type 1 deficiency syndrome. In one embodiment, the movement disorder is paroxysmal movement disorder.

Also provided is the use of a precursor of propionyl-CoA in the manufacture of a medicament for treating and/or preventing a movement disease, disorder or condition.

### BRIEF DESCRIPTION OF THE DRAWINGS

FIG. 1 illustrates the number of total paroxysmal manifestations in GLUT1-DS patients during the four phases of the study (baseline, treatment withdrawal, and resumption of treatment) of 2 months each. A significant reduction of non-epileptic paroxysmal manifestations was observed when patients were treated with triheptanoin for 2 months (*p<0.05) and when patients resumed treatment following withdrawal. Error bars represent standard error of mean (SEM).

FIG. 2 illustrates the changes in Pi/PCr ratio from f-MRS studies during the three phases of the study (baseline, treatment and withdrawal). During baseline, f-MRS showed an abnormal brain energy profile in GLUT1-DS patients with no change in Pi/PCr ratio during visual stimulation. After 2 months of treatment with triheptanoin, the profile was corrected and we observed an increase in Pi/PCr ratio during visual stimulation with a decrease during recovery (p=0.021). Error bars represent SEM of within-subject differences using the method of Morey.

### DETAILED DESCRIPTION OF THE INVENTION

The present invention provides a method of treating and/or preventing a movement disease, disorder, or condition, wherein said method comprises the step of administering a therapeutically effective amount of at least one precursor of propionyl-CoA to a subject in need thereof. In some embodiments, the subject is a human subject.

Also provided herein is a precursor of propionyl-CoA for treating and/or preventing a movement disease, disorder, or condition.

Also provided is the use of a precursor of propionyl-CoA in the manufacture of a medicament and/or food substance for treating and/or preventing a movement disease, disorder or condition.

As described herein, precursors of propionyl-CoA can be administered for the treatment of movement disorders. Movement disorders capable of being treated with the compositions and methods of the present invention can include, without limitation, dyskinesia, dystonia, ataxia, myoclonus, dysarthria, chorea, tremors, and spasticity. In some embodiments, the dyskinesia is paroxysmal exercise-induced dyskinesia.

In certain embodiments, the movement disease, disorder or condition is associated with a glucose transporter type 1 deficiency syndrome. Subjects with GLUT1-DS commonly present complex movement disorders, which can be characterized by dyskinesia, ataxia, dystonia, and chorea. These disorders can be continuous and/or paroxysmal and can fluctuate in response to different environmental stressors. The most frequent stressors are fasting, infections, exercise, and anxiety or other emotions. Pons et al. (Pons et al., 2010, Mov Disord. 25: 275-281) listed the most frequent movement disorders in 57 GLUT1-DS patients: gait disturbances such as ataxia with/without spasticity (89%), action limb dystonia (86%), chorea (75%), cerebellar action tremor (70%), non-epileptic paroxysmal events (28%), dyspraxia (21%), and myoclonus (16%).

In an exemplary embodiment, the movement disease, disorder or condition is a paroxysmal movement disorder. Paroxysmal movements can include, without limitation, myoclonic jerks, stiffening, and dystonic posturing. Paroxysmal choreoathetosis with spasticity, previously known as dystonia type 9 (DYT9), and PED, previously known as dystonia type 18 (DYT18), are now recognized to be part of the phenotypic spectrum of GLUT1-DS. Other paroxysmal events that can be observed include weakness, lethargy, somnolence, sleep disturbances, migraines, writer's cramp, parkinsonism, dyspraxia, and non-kinesigenic dyskinesia.

As described herein, the present invention provides methods of treating and/or preventing movement disorders, diseases, and conditions with precursors of propionyl-CoA. Precursors of propionyl-CoA generally include a substance from which propionyl-CoA can be formed by one or more metabolic reactions taking place within the body. Typical examples of precursors of propionyl-CoA are odd-medium-chain fatty acids, in particular the seven-carbon fatty acid, triheptanoin (triheptanoyl-glycerol), heptanoate, C5 ketone bodies (e.g. β-ketopentanoate (3-ketovalerate), and β-hydroxypentanoate (3-hydroxyvalerate)).

The examples of precursors of propionyl-CoA described above include the compounds themselves, as well as their salts, prodrugs, solvates, if applicable. Examples of prodrugs include esters, oligomers of hydroxyalkanoate such as oligo (3-hydroxyvalerate) and other pharmaceutically acceptable derivatives, which, upon administration to an individual, are capable of providing propionyl-CoA. A solvate refers to a complex formed between a precursor of propionyl-CoA described above and a pharmaceutically acceptable solvent. Examples of pharmaceutically acceptable solvents include water, ethanol, isopropanol, ethyl acetate, acetic acid, and ethanolamine.

In certain embodiments, the at least one precursor of propionyl-CoA is an uneven-chain fatty acid and more preferably, a seven-carbon fatty acid. In other preferred embodiments, the at least one precursor of propionyl-CoA is a triglyceride and more preferably a triglyceride of an uneven chain fatty acid. In other embodiments, the at least one precursor of propionyl-CoA is a phospholipid comprising one or two uneven chain fatty acid(s). In other preferred embodiments, the at least one precursor of propionyl-CoA is a C5 ketone body.

In certain embodiments, the precursor of propionyl-CoA is an uneven chain fatty acid. The invention also includes within its scope esters of uneven chain fatty acids. It will be appreciated by a person of skill in the art that an uneven chain fatty acid may also be referred to as an odd-carbon number fatty acid. In some embodiments, the uneven chain fatty acid is selected from the group consisting of propionic acid, pentanoic acid, heptanoic acid, nonanoic acid and undecanoic acid.

One practical dietary source of propionyl-CoA is triheptanoin. As will be appreciated by one skilled in the art, the invention includes within its scope the triheptanoin compound itself, as well as salts, prodrugs, analogues, derivatives, substituted, unsaturated, branched forms, or other uneven chain fatty acids and derivatives thereof, if applicable. After intestinal hydrolysis of triheptanoin, heptanoate is absorbed in the portal vein. In the liver, it is partially converted to the C5 ketone bodies β-ketopentanoate (3-ketovalerate), and β-hydroxypentanoate (3-hydroxyvalerate). The C5-ketones bodies are also precursors of propionyl-CoA in peripheral tissues. Thus, after ingestion of triheptanoin, peripheral tissues receive two precursors of propionyl-CoA, i.e., heptanoate and C5-ketone bodies. As described in the Examples, the clinical response following triheptanoin administration was associated with the significant production of C5-ketone bodies and the normalization of f-MRS bioenergetics profile during brain activation. Accordingly, triheptanoin represents a particularly beneficial source of C5-ketone bodies useful in the treatment of the movement diseases, disorders, and conditions. Thus, in an exemplary embodiment, the precursor of propionyl-CoA is triheptanoin. In other exemplary embodiments, the precursor of propionyl-CoA is heptanoic acid or heptanoate.

Triheptanoin is a triglyceride made by the esterification of three n-heptanoic acid molecules and glycerol. In regard to therapy, the terms heptanoic acid, heptanoate, and triheptanoin may be used interchangeably in the following description. Also, it will be understood by one skilled in the art that heptanoic acid, heptanoate, and triheptanoin are exemplary precursors of propionyl-CoA of the invention. Substituted, unsaturated, or branched heptanoate, as well as other modified seven-carbon fatty acids can be used without departing from the scope of the invention.

Precursors of propionyl-CoA of the present invention can be administered orally, parenterally, or intraperitoneally. Preferably, it can be administered via ingestion of a food substance containing a precursor of propionyl-CoA such as triheptanoin at a concentration effective to achieve therapeutic levels. Alternatively, it can be administered as a capsule or entrapped in liposomes, in solution or suspension, alone or in combination with other nutrients, additional sweetening and/or flavoring agents. Capsules and tablets can be coated with sugar, shellac and other enteric agents as is known. Typically medicaments according to the invention comprise a precursor of propionyl-CoA, together with a pharmaceutically-acceptable carrier. A person skilled in the art will be aware of suitable carriers. Suitable formulations for administration by any desired route may be prepared by standard methods, for example by reference to well-known text such as Remington; The Science and Practice of Pharmacy.

Typically, pharmaceutical compositions according to the invention comprise at least one precursor of propionyl-CoA together with a pharmaceutically-acceptable carrier, diluent or excipient. In particularly forms, the pharmaceutical composition is dietary formulation or a nutritional supplement and it will be that according to these embodiments, the therapeutic agent may be food-grade or be a constituent of a formulation which is food grade.

By “pharmaceutically-acceptable carrier, diluent or excipient” is meant a solid or liquid filler, diluent or encapsulating substance that may be safely used in systemic administration. Depending upon the particular route of administration, a variety of carriers, well known in the art may be used. These carriers may be selected from a group including, starches, cellulose and its derivatives, malt, gelatine, talc, calcium sulfate, vegetable oils, synthetic oils, polyols, alginic acid, phosphate buffered solutions, emulsifiers, isotonic saline and salts such as mineral acid salts including hydrochlorides, bromides and sulfates, organic acids such as acetates, propionates and malonates and pyrogen-free water.

Any safe route of administration may be employed for providing a patient with the at least one precursor of propionyl-CoA containing composition of the invention. For example, enteral, oral, rectal, parenteral, sublingual, buccal, intravenous, intra-articular, intra-muscular, intra-dermal, subcutaneous, inhalational, intraocular, intraperitoneal, intracerebroventricular, transdermal and the like may be employed. Preferably, it can be administered via ingestion of a food substance containing triheptanoin at a concentration effective to achieve therapeutic levels. Alternatively, it can be administered as a capsule or entrapped in liposomes, in solution or suspension, alone or in combination with other nutrients, additional sweetening and/or flavoring agents. Capsules and tablets can be coated with shellac and other enteric agents as is known.

Dosage forms include tablets, dispersions, suspensions, injections, solutions, syrups, oils troches, capsules, suppositories, aerosols, transdermal patches and the like. These dosage forms may also include injecting or implanting controlled releasing devices designed specifically for this purpose or other forms of implants modified to act additionally in this fashion. Controlled release of the therapeutic agent may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, the controlled release may be effected by using other polymer matrices, liposomes and/or microspheres.

Compositions of the present invention suitable for enteral, intraperitoneal, oral or parenteral administration may be presented as discrete units such as capsules, sachets or tablets each containing a pre-determined amount of the therapeutic agent of the invention, as a powder or granules or as a solution or a suspension in an aqueous liquid, a non-aqueous liquid, an oil-in-water emulsion or a water-in-oil liquid emulsion. Preferably, administration of the agent of the invention is by way of oral administration. Such compositions may be prepared by any of the methods of pharmacy but all methods include the step of bringing into association one or more agents as described above with the carrier which constitutes one or more necessary ingredients. In general, the compositions are prepared by uniformly and intimately admixing the agents of the invention with liquid carriers or finely divided solid carriers or both, and then, if necessary, shaping the product into the desired presentation.

The above compositions may be administered in a manner compatible with the dosage formulation, and in such amount as is pharmaceutically-effective. The dose administered to a patient, in the context of the present invention, should be sufficient to effect a beneficial response in a patient over an appropriate period of time. The quantity of agent(s) to be administered may depend on the subject to be treated inclusive of the age, sex, weight and general health condition thereof, factors that will depend on the judgement of the practitioner.

It will be appreciated by the skilled artisan that a therapeutically effective amount is a sufficient amount of at least one precursor of propionyl-CoA to substantially alleviate, ameliorate, reduce and/or eliminate one or more symptoms of a movement disease, disorder, or condition.

According to some embodiments of the present invention, administration of the at least one precursor of propionyl-CoA provides a statistically significant therapeutic effect for the treatment of the movement disease, disorder, or condition. In one embodiment, the statistically significant therapeutic effect is determined based on one or more standards or criteria provided by one or more regulatory agencies in the United States, e.g., FDA or other countries. In another embodiment, the statistically significant therapeutic effect is determined based on results obtained from regulatory agency approved clinical trial set up and/or procedure.

In some embodiments, the statistically significant therapeutic effect is determined based on data with an alpha value of less than or equal to about 0.05, 0.04, 0.03, 0.02 or 0.01. In some embodiments, the statistically significant therapeutic effect is determined based on data with a confidence interval greater than or equal to 95%, 96%, 97%, 98% or 99%. In some embodiments, the statistically significant therapeutic effect is determined based on data with a p value of less than or equal to about 0.05, 0.04, 0.03, 0.02 or 0.01. In some embodiments, the statistically significant therapeutic effect is determined on approval of Phase III clinical trial of the compositions and methods provided by the present invention, e.g., by FDA in the US.

In general, statistical analysis can include any suitable method permitted by a regulatory agency, e.g., FDA in the US or China or any other country. In some embodiments, statistical analysis includes non-stratified analysis, log-rank analysis, e.g., from Kaplan-Meier, Jacobson-Truax, Gulliken-Lord-Novick, Edwards-Nunnally, Hageman-Arrindel and Hierarchical Linear Modeling (HLM) and Cox regression analysis.

Administration of a propionyl-CoA precursor, as described herein, results in a reduction in paroxysmal manifestations associated with GLUT1-DS. In some embodiments, the number of paroxysmal manifestations following administration of one or more propionyl-CoA precursors is reduced by at least about 5% (e.g., by at least about 10%, 15%, 20%, 25%, 30%, 35%, 40%, 50%, 60%, 70%, 80%, 90%, or 95%). Paroxysmal manifestations associated with GLUT1-DS can include, without limitation, dyskinesia, myoclonic jerks, stiffening, tremors, dystonic movements, dystonic posturing, and choreic movements. In some embodiments, the dyskinesia is paroxysmal exercise-induced dyskinesia. In some embodiments, administration of one or more propionyl-CoA precursors results in a statistically significant reduction in paroxysmal manifestations associated with GLUT1-DS. In certain exemplary embodiments, the propionyl-CoA precursor is triheptanoin.

As is understood in the art, dystonia is a neurological movement disorder in which sustained muscle contractions cause twisting and repetitive movements or abnormal postures. The movements may resemble a tremor. Meanwhile, dyskinesia refers to abnormal involuntary movements. In some embodiments, administration of one or more propionyl-CoA precursors results in a reduction in dystonic or dyskinetic events associated with GLUT1-DS. In some embodiments, the number of dystonic or dyskinetic events following administration of one or more propionyl-CoA precursors is reduced by at least about 5% (e.g., by at least about 10%, 15%, 20%, 25%, 30%, 35%, 40%, 50%, 60%, 70%, 80%, 90%, or 95%). Dystonic events may include anismus, cervical dystonia, blepharospasm, oculogyric crisis, oromandibular dystonia, laryngeal dystonia, and focal hand dystonia. In some embodiments, administration of one or more propionyl-CoA precursors results in a statistically significant reduction in dystonic or dyskinetic events associated with GLUT1-DS. In certain exemplary embodiments, the propionyl-CoA precursor is triheptanoin.

In some embodiments, the reduction in paroxysmal manifestations and/or dystonic or dyskinetic events is determined by measuring the quantity and/or frequency of paroxysmal manifestations and/or dystonic or dyskinetic events occurring during a period of time (i.e., a time window) prior to treatment, i.e., a “baseline phase” or “baseline period” and comparing that value to the quantity and/or frequency (e.g., total number, average number per day, average number per week, average number per month, frequency, etc.) of paroxysmal manifestations and/or dystonic or dyskinetic events occurring over a period of time during treatment with one or more propionyl-CoA precursors, i.e., “treatment period”.

In some embodiments, the baseline phase is a time period of 1 day, 1 week, 2 weeks, 3 weeks, 4 weeks, 6 weeks, 8 weeks, 12 weeks, 16 weeks, 20 weeks, or 24 weeks, and any time periods in between, prior to administration of one or more propionyl-CoA precursors. In some embodiments, the baseline phase is a time period of 1 month, 2 months, 3 months, 4 months, 6 months, 8 months, 12 months, 16 months, 20 months, or 24 months, and any time periods in between, prior to administration of one or more propionyl-CoA precursors. In some embodiments, the treatment period is measured 1 day, 1 week, 2 weeks, 3 weeks, 4 weeks, 6 weeks, 8 weeks, 12 weeks, 16 weeks, 20 weeks, or 24 weeks, and any time periods in between, after starting treatment with one or more propionyl-CoA precursors. In some embodiments, the treatment period is measured 1 month, 2 months, 3 months, 4 months, 6 months, 8 months, 12 months, 16 months, 20 months, or 24 months, and any time periods in between, after starting treatment with one or more propionyl-CoA precursors. In certain exemplary embodiments, the propionyl-CoA precursor is triheptanoin.

In some embodiments, the reduction in paroxysmal manifestations and/or dystonic or dyskinetic events is measured by determining the number of paroxysmal manifestations and/or dystonic or dyskinetic events occurring in a single patient. In preferred embodiments, the reduction in paroxysmal manifestations and/or dystonic or dyskinetic events is measured by determining the average number of paroxysmal manifestations and/or dystonic or dyskinetic events occurring in a group of patients.

The terms “effective amount”, “amount effective to”, or “therapeutically effective amount” in the context of the present invention refers to an amount of a propionyl-CoA precursor which reduces paroxysmal manifestations and/or dystonic or dyskinetic events (e.g., to produce a statistically significant reduction) in patients. The “effective amount” can be readily determined, in accordance with the invention, by administering to a plurality of tested subjects various amounts of the propionyl-CoA precursor and then plotting the physiological response (for example the reduction in paroxysmal manifestations and/or dystonic or dyskinetic events, or the improvement following treatment determined using the CGI-I scale) as a function of the amount of the propionyl-CoA precursor administered. Alternatively, the effective amount may also be determined, at times, through experiments performed in appropriate animal models and then extrapolating to human beings using one of a plurality of conversion methods.

In some embodiments, administration of a propionyl-CoA precursor results in a reduction of clinical symptoms associated with GLUT1-DS mediated movement disorders. In a preferred embodiment, the clinical symptoms include paroxysmal movements. The phrase “reduction in clinical symptoms” means, but is not limited to, the frequency in the incidence of at least one clinical symptom following administration of at least one propionyl-CoA precursor in a group of subjects is at least 10%, preferably 20%, more preferably 40%, and even more preferably 60% lower prior to administration of the at least one propionyl-CoA precursor. In certain exemplary embodiments, the propionyl-CoA precursor is triheptanoin.

The present invention includes within its scope a therapeutic amount of at least one precursor of propionyl-CoA is less than 100% of dietary caloric intake and preferably, within a range from between about 5% and about 90%, within a range from between about 15% and about 80%, within a range from between about 20% and about 60%, within a range from between about 25% and 50% and within a range from between about 30% and about 40%.

In some embodiments, the at least one precursor of propionyl-CoA is provided to the animal in an amount comprising at least about 5%, at least about 10%, at least about 15%, at least about 20%, at least about 20.5%, at least about 21%, at least about 21.5%, at least about 22%, at least about 22.5%, at least about 23%, at least about 23.5%, at least about 24%, at least about 24.5% at least about 25%, at least about 25.5%, at least about 26%, at least about 26.5%, at least about 27%, at least about 27.5%, at least about 28%, at least about 28.5%, at least about 29%, at least about 29.5%, at least about 30%, at least about 30.5%, at least about 31%, at least about 31.5%, at least about 32%, at least about 32.5%, at least about 33%, at least about 33.5%, at least about 34%, at least about 34.5%, at least about 35%, at least about 35.5%, at least about 36%, at least about 36.5%, at least about 37%, at least about 37.5%, at least about 38%, at least about 38.5%, at least about 39%, at least about 39.5%, at least about 40%, at least about 40.5%, at least about 41%, at least about 41.5%, at least about 42%, at least about 42.5%, at least about 43%, at least about 43.5%, at least about 44%, at least about 44.5%, at least about 45%, at least about 45.5%, at least about 46%, at least about 46.5%, at least about 47%, at least about 47.5%, at least about 48%, at least about 48.5%, at least about 49%, at least about 49.5%, at least about 50%, at least about 55%, at least about 60%, about at least about 70%, at least about 80%, at least about 90% or more of the dietary caloric intake.

Propionyl-CoA precursors and related compositions are formulated so as to allow the active ingredients contained therein to be bioavailable upon administration of the composition to a subject. Compositions that will be administered to a subject or patient usually take the form of one or more dosage units, where for example, a tablet or capsule (e.g., gel capsule) may be a single dosage unit, and a container may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). In certain aspects, the composition to be administered contains a therapeutically effective amount of a propionyl-CoA precursor, for treatment of a disease or condition of interest, e.g., a movement disease, disorder or condition.

In some embodiments, a unit dosage comprises about or at least about 2 g to about 150 g, or about 2 g, 3 g, 4 g, 5 g, 10 g, 15 g, 20 g, 25 g, 30 g, 35 g, 40 g, 45 g, 50 g, 55 g, 60 g, 65 g, 70 g, 75 g, 80 g, 90 g, 95 g, 100 g, 125 g or 150 g, or more of a propionyl-CoA precursor (e.g., triheptanoin).

The frequency of administration of the compositions described herein may vary from once-a-day (QD) to twice-a-day (BID) or thrice-a-day (TID), etc., the precise frequency of administration varying with, for example, the patient's condition, the dosage, etc.

In certain embodiments a dosage is calculated by the weight of the subject. According to the World Health Organization (WHO), boys from birth to 5 years of age range in mass from approximately 2 kg to 30 kg. Boys of 5 years to 10 years of age range in mass from approximately 10 kg to 50 kg. Girls from birth to 5 years of age range in mass from approximately 2 kg to 30 kg. Girls of 5 years to 10 years of age range in mass from approximately 10 kg to 52 kg. See, for example, the WHO Growth Standards, hereby incorporated by reference.

In certain embodiments, the dosage of the propionyl-CoA precursor, e.g., triheptanoin, is from about 2-4 grams/kg for infants, 1-3 grams/kg for young children (e.g., prepubescent or pubescent), or about 1-2 grams/kg for adolescents (e.g., post-pubescent) and adults. In specific embodiments, the dosage ranges from about 1-6, 1-2, 2-3, 3-4, 4-5, or 5-6 grams/kg for infants, 0.5-4, 0.5-1, 1-1.5, 1.5-2, 2-2.5, 2.5-3, 3-3.5, or 3.5-4 grams/kg for young children, or about 0.5-4, 0.5-1, 1-1.5, 1.5-2, 2-2.5, 2.5-3, 3-3.5, or 3.5-4 grams/kg for adolescents and adults.

In some embodiments, the unit dosage is the desired daily dosage (e.g., grams/kg) multiplied by the average weight of the subject group, and optionally divided by times per day for administration. For example, in some embodiments, the unit dosage for infants is 2-4 grams/kg multiplied by an average infant's weight, and optionally divided by one, two, three, four, five or six for daily administration. In particular embodiments, the unit dosage for young children through school age is 1-2 grams/kg multiplied by an average young child's weight, and optionally divided by one, two, three, four, five or six for daily administrations. In some embodiments, the unit dosage for adolescents and adults is about 1 grams/kg multiplied by an average adolescent's or adult's weight, and optionally divided by one, two, three, or four for daily administration. In some embodiments the unit dosage volume is in milliliters or liters.

In some embodiments, the propionyl-CoA precursor (e.g., triheptanoin) is provided in solution and/or oil from between about 0.25 g/mL (i.e., 0.25 g per cc) to about 2 g/mL (i.e., 0.25 g per cc). In certain embodiments, the odd-chain fatty acid source (e.g., triheptanoin) is provided (e.g., in solution and/or oil) at about 0.25 g/mL, 0.5 g/mL, 0.75 g/mL, 1 g/mL, 1.25 g/mL, 1.5 g/mL, 1.75 g/ml or 2 g/mL.

In some embodiments, the propionyl-CoA precursor (e.g., triheptanoin) is administered at about 1 to about 10 grams/kg/24 hours, about 1 to about 5 grams/kg/24 hours or about 1 to about 2 grams/kg/24 hours. In some embodiments, the propionyl-CoA precursor (e.g., triheptanoin) is administered at about 2-4 grams/kg/24 hours for infants. In some embodiments, the propionyl-CoA precursor (e.g., triheptanoin) is administered at about 2, 3 or 4 grams/kg/24 hours for infants. In certain embodiments, the propionyl-CoA precursor (e.g., triheptanoin) is administered at about 1-3 grams/kg/24 hours for children through school age. In some embodiments, the propionyl-CoA precursor (e.g., triheptanoin) is administered at about 1, 2, or 3 grams/kg/24 hours for children through school age. In some embodiments the propionyl-CoA precursor (e.g., triheptanoin) is administered at about 1-2 grams/kg/24 hours for adolescents and adults. In some embodiments, the propionyl-CoA precursor (e.g., triheptanoin) is administered at about 1 or 2 grams/kg/24 hours for adolescents and adults.

Based on the suitable dosage, the propionyl-CoA precursor (e.g., triheptanoin) can be provided in various suitable unit dosages. For example, a propionyl-CoA precursor can comprise a unit dosage for administration of one or multiple times per day, for 1-7 days per week. Such unit dosages can be provided as a set for daily, weekly and/or monthly administration.

In some embodiments, the propionyl-CoA precursor (e.g., triheptanoin) is administered about six times a day, about five times a day, about four times a day, about three times a day, about twice a day, or about once per day.

In certain embodiments, the daily dosage is divided into 1 to 6 daily dosages, 1 to 5 daily dosages, or 1 to 4 daily dosages. In some embodiments, the daily dosage is divided into 1 to 4 daily doses, 1 to 5 daily doses, or 1 to 6 daily doses of 2, 5, 10, 15, 20, 25, 30, 35, 40 or 50 cc. In some embodiments, the daily dosage is divided into 4 daily doses of 15 cc to 20 cc for an adult. In specific embodiments, the propionyl-CoA precursor (e.g., triheptanoin) is provided in 1 g/mL and the daily dosage of 1 g/kg/day is divided into 4 daily dosages.

In some embodiments, the propionyl-CoA precursor (e.g., triheptanoin) is administered for one week, two weeks, one month, two months, six months, twelve months, eighteen months, or more.

In some embodiments, the precursor of propionyl-CoA is administered in the absence of a ketogenic diet.

By “ketogenic diet” is meant a high fat and low carbohydrate and protein diet. Typically, a ketogenic diet contains a 3:1 to 4:1 ratio by weight of fat to combined protein and carbohydrate. A ketogenic diet may refer to a classical ketogenic diet comprising predominantly natural fats (inclusive of normal dietary fats and suitably long-chain triglycerides) or a ketogenic diet comprising predominantly medium chain triglycerides and suitably, even medium chain triglycerides.

In the context of the present invention, by “absence of a ketogenic diet” is meant a dietary intake which does not have a higher than normal fat content compared to carbohydrate and protein. In some preferred embodiments, an “absence of a ketogenic diet” is a diet is which the ratio by weight of fat to combined protein and carbohydrate is less than 3:1, and may be 2:1, 1:1, 0.5:1 or a ratio where the fat content is even lower, or where fat is absent. The ketogenic diet may be a classical ketogenic diet or a medium chain triglyceride ketogenic diet as hereinbefore described.

This invention is further illustrated by the following example that should not be construed as limiting. The contents of all references, patents, and published patent applications cited throughout this application are incorporated herein by reference for all purposes.

### EXAMPLES

### Example 1: Clinical and Metabolic Response to Triheptanoin

This example describes an open-label pilot study with four phases of 2 months each (baseline, treatment withdrawal, and resumption of treatment) in eight GLUT1-DS patients (7-47 years old) with non-epileptic paroxysmal manifestations.

### Methods:

Participants were enrolled in an interventional clinical protocol. Four children and four adults with GLUT1-DS were enrolled. They had a chronic history of non-epileptic paroxysmal episodes, associated for two patients with a mild cognitive deficit. All patients were on a normal diet prior to their enrollment. As noted above, the study was divided into four phases of 2 months each (baseline, treatment, withdrawal, and resumption of treatment). A trained dietitian determined patients' caloric intake and adapted the daily menus so the diets remained isocaloric when triheptanoin was introduced.

During the treatment phase, patients ingested 1 g/kg body-weight of triheptanoin per day, divided in 3 to 4 intakes during meals. A comprehensive diary was kept for each patient to record all motor and non-motor paroxysmal events—including dystonia, tremor, speech disorder, stiffness, paresis, headaches, confusion, and lethargy. At each visit, patients were evaluated with a six minute walk test (6MWT), a nine-hole pegboard test (NHPT), the clinical global impression-improvement scale (CGI-I) and a fatigue severity scale (KFFS or visual analogue scale for patients <15 years old). Blood samples were collected after an overnight fast for standard analyses, and the measurement of plasma C3-carnitine and C5-ketone bodies (Mochel et al., 2005, Molecular Genetics and Metabolism 84: 305-312).

Functional ³¹P-NMR spectroscopy (f-MRS) was performed at 3 T at the end of each study phase in patients >15 years old (n=5). Data were collected for 4 minutes at rest, 8 minutes during visual activation, 8 minutes after stimulation, and analyzed as described (Adanyeguh et al., 2015, Neurology 84: 490-495 and Mochel et al., 2012, Mov Disord. 27: 907-910). The ratio of Pi/PCr was calculated to determine the brain response to cortical activation.

Paired t-tests were used for plasma analyses before and after treatment. For clinical parameters, Friedman tests were used to test the global hypothesis that all study phases were equal. If significant, Wilcoxon signed-rank tests were applied for pair-wise phase comparisons with an alpha of 0.05. For the Pi/PCr ratio, repeated measures ANOVA were used to test the global hypothesis that all time points—rest, activation and recovery—were equal. If significant, paired t-tests were applied for pair-wise time comparisons with an alpha of 0.05.

### Results—Clinical Response:

Triheptanoin was well tolerated in all patients. Nonetheless, two patients were considered not compliant with the study as they consumed less than 50% of the recommended dose of triheptanoin and they (or their legal guardians) regularly omitted to fill the patient diary. Accordingly, data analysis was performed in six patients out of the eight initially enrolled.

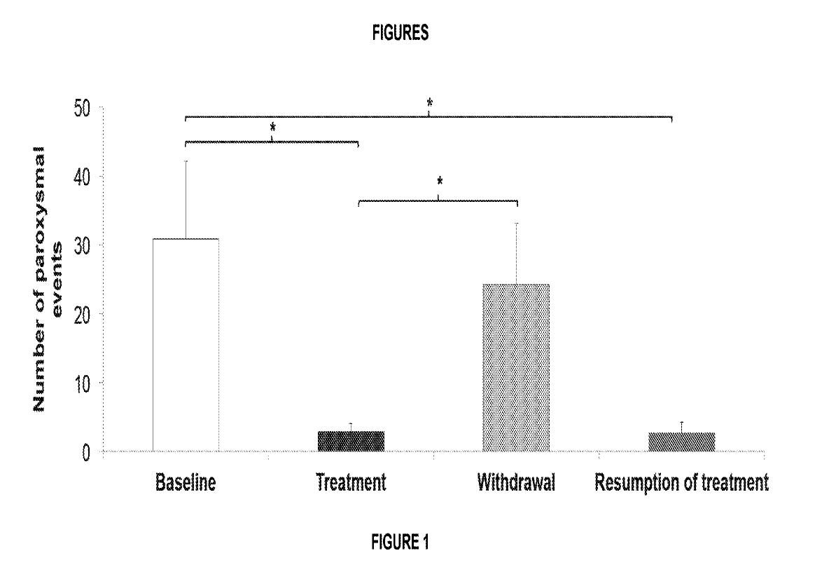

During the baseline phase, GLUT1-DS patients experienced an average of 31 paroxysmal manifestations (±28, 10-85), including 16 dystonic events (±19, 1-54). When treated with triheptanoin for 2 months, paroxysmal manifestations dropped to an average of 3 (±3, 0-7), including 2 dystonic events (±3, 0-7) (p=0.028, FIG. 1). On the CGI-I scale, all patients reported a clear improvement when treated (“much improved”). Their fatigue score tended to improve on triheptanoin, although it did not reach significance; their performance during the 6WMT and NHPT was unchanged (data not shown). During the withdrawal phase, patients experienced an average of 24 paroxysmal manifestations (±22, 5-63), including 13 dystonic events (±15, 1-40) (p=0.043, FIG. 1). On the CGI-I scale, five out of six patients reported a clear worsening during withdrawal (“much worse”). Their fatigue score worsened but it did not impact their performance on the 6WMT and NHPT (data not shown). Paroxysmal manifestations again dropped significantly after treatment was resumed.

### Results—Metabolic Response:

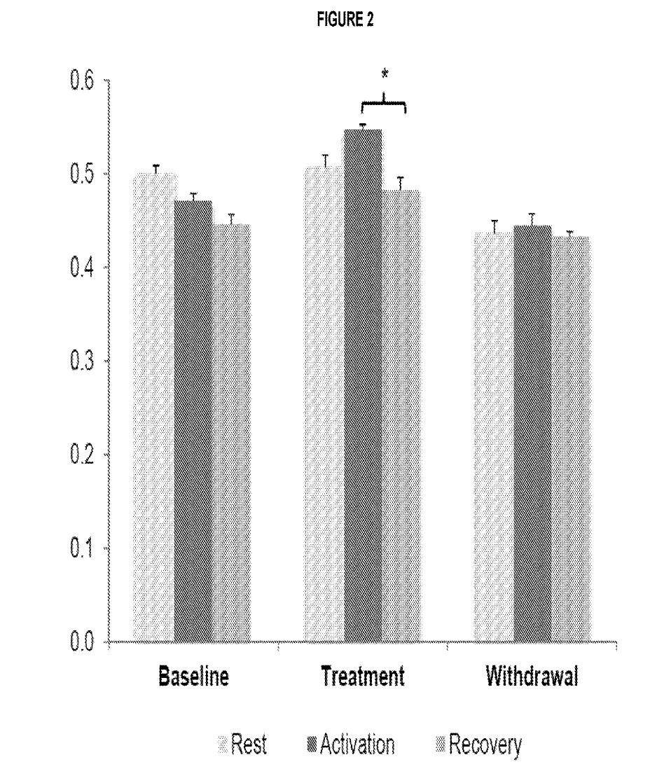

Compared to baseline, a significant increase of plasma C3-carnitine (p=0.026) and C5-ketone bodies (p=0.008) on triheptanoin was observed, reflecting its proper metabolism in the six compliant GLUT1-DS patients. Conversely, the levels of triheptanoin metabolites were unchanged in the two non-compliant patients. During baseline, f-MRS showed no change in Pi/PCr ratio during brain activation in GLUT1-DS patients (FIG. 2), unlike what was reported in healthy individuals.

After 2 months on triheptanoin, the bioenergetics profile normalized and repeated measures ANOVA were significant for Pi/PCr ratio (p=0.014). An increase in Pi/PCr ratio during visual stimulation and a decrease during recovery using the Bonferroni corrected paired t-tests (p=0.021, FIG. 2) was observed. Increased Pi/PCr ratio during brain activation reflected a proportional elevation of ADP, allowing increased mitochondrial ATP production with triheptanoin. After treatment withdrawal, the f-MRS profile came back abnormal (FIG. 2).

The above data demonstrates that treatment with triheptanoin rapidly reduced the number of non-epileptic paroxysmal manifestations in children and adults with GLUT1-DS. This striking clinical response was associated with a significant production of C5-ketone bodies and the normalization of the f-MRS bioenergetics profile during brain activation. Despite the absence of a control group, the magnitude of the clinical effect concomitant to the metabolic responses rules out a placebo effect. This study demonstrates a sustainable clinical improvement with triheptanoin in GLUT1-DS together with a robust metabolic response using a validated biomarker of brain energy metabolism. The long-term confirmation of this data holds the promise of an alternative therapeutic approach to ketogenic diets in the treatment of GLUT1-DS.

Unless defined otherwise, all technical and scientific terms used herein have the same meaning as commonly understood to one of ordinary skill in the art to which the present application belongs. Although any methods and materials similar or equivalent to those described herein can be used in the practice or testing of the present application, representative methods and materials are herein described.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

The disclosures, including the claims, figures and/or drawings, of each and every patent, patent application, and publication cited herein are hereby incorporated herein by reference in their entireties. In the case of any conflict between a cited reference and this specification, the specification shall control. In describing embodiments of the present application, specific terminology is employed for the sake of clarity. However, the invention is not intended to be limited to the specific terminology so selected. Nothing in this specification should be considered as limiting the scope of the present invention. All examples presented are representative and non-limiting. The above-described embodiments may be modified or varied, without departing from the invention, as appreciated by those skilled in the art in light of the above teachings.

1-19. (canceled)

20. A method of treating and/or preventing a paroxysmal movement disorder in a human subject, wherein said method comprises the step of administering to the human subject a pharmaceutical composition consisting essentially of a branched amino acid.

21. The method of claim 20, wherein the pharmaceutical composition is administered orally.
